(19) United States
(12) Patent Application Publication
(10) Pub. No.: US 2005/0177893 A1
(43) Pub. Date: Aug. 11, 2005

(54) Transcription factors and methods for introduction of value-added seed traits and stress tolerance

(76) Inventors: Christopher Dale Rock, Lubbock, TX (US); Srinivas S. L. Gampala, Lubbock, TX (US)

Correspondence Address: JONES, TULLAR & COOPER, P.C., P.O. BOX 2266 EADS STATION, ARLINGTON, VA, 22202, US

(21) Appl. No.: 10/629,907

(22) Filed: Jul. 30, 2003

Related U.S. Application Data
(60) Provisional application No. 60/399,565, filed on Jul. 30, 2002.

(51) Int. Cl. A01H 1/00; C12N 15/82; C12Q 1/68

(52) U.S. Cl. 800/278; 435/468

(57) ABSTRACT

A variety of stress-related traits in plants are enhanced by the synergistic use of ABI-5 and Viviparous-1 (VP-1) like transcription factors to regulate abscisic acid (ABA)-inducible gene expression. Using these transcription factors in novel formulations, as shown by examples, will confer value-added traits to transgenic plants, including, but not limited to, drought and salt tolerance, viability under stress, and enhance nutrient reserves.

### CROSS-REFERENCE TO RELATED APPLICATION

This application claims the benefit, under 35 U.S.C. 119(e), of U.S. Provisional Application No. 60/399,565 filed Jul. 30, 2002, the contents of which are incorporated herein by reference.

### BACKGROUND OF THE INVENTION

1. Field of the Invention

A variety of stress-related traits in plants are enhanced by the synergistic use of ABI-5 and Viviparous-1 (VP-1) like transcription factors to regulate abscisic acid (ABA)-inducible gene expression.

2. Description of Related Art

The growth in the world's population combined with a general increase in global prosperity is creating an increasing demand for food and sustainable agriculture. It is estimated that the world's population will increase by 80% to 10.8 billion people by 2050, with a concomitant decrease in arable land of 20%. For example, rice is the staple food for two-thirds of the world's population and is the primary cereal crop in the world, with worldwide production in 2000 of 600 million tons. 92% of the world's rice is produced in Asia, and 40% of the cultivated area is rain-fed and experiences environmental stress, with losses estimated at 200 million tons/yr. A worthwhile future can only be guaranteed through sustainable agriculture and a protective relationship with nature.

Yield enhancement to increase crop production is one of the essential strategies to meet the demand for food by the growing population. In order to supply the world's population in 20 years' time with enough to eat, today's food production will have to be doubled on a third less land and water. For example, due to traditional rice breeding advances, with which germplasm from wild relatives was transferred to cultivated strains, production of rice doubled between 1966 and 1990, but it is estimated that production must increase 60% by 2025 to meet demand. The rate at which growers have been able to further improve crop productivity has declined as improved farming practices have become more fully implemented around the world, as land in developed countries available for conversion to farming has declined, and as concerns about the environmental impact of farming have increased.

While the rate of yield increases from hybrids has slowed in the last two decades, the application of biotechnology and genomics is dramatically increasing innovation in the agricultural and seed industries. Biotechnology as a means of sustainable agriculture is a crucial component to meeting the challenges posed by the interrelated global issues of poverty, hunger, population growth, and environmental degradation in the twenty-first century. Biotechnology enables gene-by-gene analysis and enhancement of crops and is augmenting traditional breeding by enabling faster, targeted development of performance-enhancing traits. These traits currently are designed to create higher-quality animal feed and in the future are expected to include nutritional benefits for humans.

Growers have rapidly adopted the first generation of genetically engineered seed traits, with significant numbers of acres planted. The number of global planted acres of herbicide-tolerant and insect-resistant crops grew from less than 5 million acres in 1995 to approximately 120 million acres in 1999. Despite this rapid growth, the total number of acres covered currently represents only a small fraction of the approximately 3 billion acres of crops cultivated worldwide. Additional growth will come from further adoption of currently available traits and the development of new input and output traits.

Improvement of crop plants for a variety of traits, including disease and pest resistance, adaptation to abiotic stresses, and grain quality improvements such as oil, starch or protein composition, has been achieved by introducing new or modified genes into plant genomes. It has recently been shown that the “Green Revolution” of the 1960s that resulted in large increases in wheat yields was due to adoption of varieties that contain dominant alleles of a putative transcription factor involved in plant hormone signaling. Transcription factors control virtually all significant plant traits, including yield, disease resistance, freezing and drought protection, as well as the production of chemicals and proteins used as pharmaceuticals, nutriceuticals and consumer products, by coordinate regulation of multiple target genes whose functions in many cases are not yet known.

The expression of target transgenes and endogenous genes is controlled through a complex set of protein/DNA and protein/protein interactions. Promoters and enhancers can impart patterns of expression that are either constitutive or limited to specific tissues or times during development, or in response to environmental stimuli. There are limitations in the types of expression achievable using existing promoters for transgene expression. One limitation is in the expression level achievable. It is difficult to obtain traits that require relatively high expression of an introduced gene, due to limitations in promoter strength. A second limitation is that the pattern of expression conferred by the particular promoter employed is inflexible in that the same promoter-dependent pattern of expression is conferred from generation to generation. It is desirable to have the ability to regulate trait-conferring transgene expression differently in successive generations. One example would be a trait that has a side effect of being detrimental to seed quality, but which is desired for use in fodder. In this case, it would be desirable to carry the trait-conferring transgene in an inactive state in separate breeding stocks.

Plants are sessile and therefore must perpetually develop in response to their changing environment. Plants have evolved complex, integrated, and overlapping signaling pathways to maintain a plastic growth habit in response to stresses such as drought, salt, cold, as well as hormonal cues such as abscisic acid (ABA). ABA mediates a myriad of physiological processes in growth and development, including cell division, water use efficiency, and gene expression during seed development and in response to environmental stresses such as drought, chilling, salt, pathogen attack, and UV light. Despite the complex multitude of physiological, molecular, genetic, biochemical, and pharmacological data that implicate ABA in stress responses, the adaptive responses to ABA and stresses, and the pathways that trigger them, are largely unknown. Seed maturation and freezing/drought/salt tolerance may have certain protective mechanisms in common, since they share the common phenomenon of dehydration stress.

It would be advantageous for genetic engineering of plants for environmental stress resistance to regulate multiple genes in a particular metabolic or response pathway via a single transgene. Cloning and overexpression of Drought Response Element Binding (DREB)/Cold Binding Factor (CBF) subfamily of the AP2-domain family of transcription factors responsible for cold-inducible gene expression has demonstrated the practical benefits of coordinated activation of uncharacterized gene sets that can confer non-specific protection to transgenic plants by up-regulation or pre-activation of stress-response pathways. The ABA-INSENSITIVE-4 gene is most closely related to the DREB/CBF subfamily of the AP2-domain family. Transgenic overexpression of the transcription factor ALFIN1 enhances expression of the endogenous MsPRP2 gene in alfalfa and improves salinity tolerance of the plants. Over-expression of a single Ca²⁺-dependent protein kinase confers both cold and salt/drought tolerance on rice plants. A multi-component transcription factor/target promoter system that regulates hormone and stress responses could be used to address the limitations of single transgene expression and tap into the natural defense systems of crop plants.

Although hundreds of ABA-regulated genes have been identified to date, many of them homologs from a broad range of species, these are likely to represent a somewhat anecdotal sampling of the full spectrum of ABA-responsive genes. Preliminary genome profiling in Arabidopsis has allowed estimation of the number of plant genes modulated by ABA, with current estimates at about 2000 genes. A number of plant gene products have been identified that may function in desiccation tolerance. The COR genes are cold-, drought-, salt-, and ABA-responsive genes whose protein products are heat stable and hydrophyllic; some COR genes have structural similarities to the late embryogenesis-abundant (LEA) proteins. LEA homologues in wheat, maize, barley, carrot, and the resurrection plant Craterostigma plantagineum are induced by ABA and dehydration stress. The exact roles of COR and LEA genes in cold and desiccation tolerance are not yet known, but there is strong evidence that they have an adaptive function in desiccation, freezing, and salt tolerance. Altered expression of ABA signal transduction genes can have beneficial effects on stress adaptation of plants.

The RY-G-box-RY regulatory element is commonly found in seed storage protein promoters and is necessary for positive regulation of seed-specific gene expression in the β-phaseolin and Em promoters. The sequences of the RY-G-box-RY elements that are found in different natural promoters have variations, but can be recognized by the presence of particular nucleotide sequences: CATGCAW (the “RY” feature) and CACGTG (the “G-box”). There is substantial diversity in the cis sequences shown to confer ABA-inducible expression. The smallest promoter units (called ABA-Response Elements; ABREs) that are both necessary and sufficient for ABA induction of gene expression appear to consist of at least two essential cis elements, one of which is usually a G-box and the other a “coupling” element.

A seed-specific regulatory factor, Viviparous-1 (VP1), was first described in 1931 and was cloned by transposon tagging in 1989. The ABA-INSENSITIVE3 (ABI3) gene of Arabidopsis is the genetic equivalent of maize VP1 and was cloned by chromosome walking. VP1/ABI3 is expressed in developing seeds and precedes ABA-inducible storage protein and late-embryogenesis-abundant (LEA) gene expression. Rice and maize protoplasts that transiently overexpress the VP1 cDNA can transactivate ABA-inducible promoters from numerous species. Similar transactivation results have been obtained in homologous transient gene expression systems with the rice VP1 and bean Pv-ALF orthologs. Remarkably, VP1 also has repressor activity towards the germination-specific alpha-amylase genes, but repression is non-cell-autonomous and requires embryo-specific factors other than ABA and VP1.

Structure/function studies with VP1 and PvALF in transient gene expression assays demonstrate that the highly conserved N-terminal acidic domain (A1, amino acids 51-163) functions as a transcriptional activator and acts in synergy with ABA. The acidic domain of VP1 is not required for germination-specific alpha-amylase gene repression. The conserved basic B2 region (aa 508-544) is required for transactivation of the ABA-inducible Em promoter and for enhancing the in vitro binding of various basic leucine zipper (bZIP) factors to their cognate targets, but not for alpha-amylase gene repression. The B3 domain (aa 632-760) binds specifically to promoter sequences required for transactivation but not to ABA-responsive cis-elements. Furthermore, the B3 domain is not required for synergistic effects of transactivation with ABA or for alpha-amylase gene repression. Pv-ALF facilitates chromatin modification of the ABA-inducible β-Phas promoter, which in turn potentiates ABA-mediated transcription. Carrot and Arabidopsis Pv-ALF orthologs can also direct ABA-inducible seed storage protein expression in leaves when expressed ectopically. The exact molecular mechanisms of ABI3/VP1/Pv-ALF are not known, but the predicted FUS3 and LEAFY COTYLEDON-2 class of regulators that control embryo maturation have a continuous stretch of more than 100 amino acids showing significant sequence similarity to the conserved B3 domains of ABI3/VP1/Pv-ALF. This suggests that ABI3, LEC2, and FUS3 may act in partially redundant pathways. The Arabidopsis genome encodes 43 members of the B3-domain family, 14 of them within the ABI3/VP1-related subfamily.

There are 81 predicted bZIP factor genes in Arabidopsis, but only one bZIP subfamily has been genetically or functionally linked to ABA response: that composed of ABI5 and its homologs, including the ABRE Binding Factors (ABFs and AREBs), Enhanced Em Level (EEL/AtbZIP12), and AtbZIP13-15, 27, and 67, which include the AtDPBFs (Arabidopsis thaliana Dc3 Promoter Binding Factors). Homologs of these genes have been characterized in sunflower and rice, where they are also correlated with ABA-, seed- or stress-induced gene expression. However, studies of bZIPs from other species have shown that in vitro binding of ABREs need not reflect action in ABA signaling in vivo. A rice homolog of ABI5, TRAB1, was identified by a two-hybrid screen using the basic domains of OsVP1 as “bait” and shown to interact with ABREs in vitro and activate ABA-inducible transcription in rice protoplasts. AREBs and ABFs both share with ABI5 three conserved charged domains (C1-C3) in their amino-halves as well as the bZIP domain and another conserved (C4) domain at the C-terminus. In vitro studies with the DPBFs and other ABI5-family members have demonstrated that this subfamily binds to G-box elements required for ABA regulation and consequently designated ABREs (ABA response elements). However, the ABI5/DPBF/ABF/AREB subfamily has a broader consensus sequence for its binding site than the other bZIP proteins in that its members tolerate variability in the ACGT core element essential to the ABRE G-box. ABI5 and its homolog EEL were shown to compete for the same binding sites in AtEm1 promoter and a model was proposed, based on single and double mutant phenotypes of altered gene expression, that EEL directly antagonized ABI5 transactivation. Analyses of transcript accumulation in abi5 mutants suggest that, similar to ABI3, ABI5 has both activator and repressor functions, but that ABI5 and ABI3 may have either synergistic or antagonistic effects on gene expression, depending on the gene. ABI5 protein accumulation is further enhanced by ABA-induced phosphorylation and resulting stabilization of the protein, at least during the early phases of germination.

New methods which genetically engineer value-added vegetative traits for stress adaptation and seed qualities by directed expression of ABA-related transcription factors would be beneficial to supply the world with the increased amounts of food needed by future generations. Simultaneously promoting coordinated regulation of multiple endogenous genes is important for stress adaptation. By overcoming the limitations of targeted gene expression and by transactivation of endogenous plant stress adaptation pathways, the volume and quality of products in environments under stress will improve.

### BRIEF SUMMARY OF THE INVENTION

There are limitations in the types of gene expression achievable using promoters for plant transgene expression. For example, the expression level achievable is often limited due to insufficient promoter strength, and the patterns of expression conferred by a particular promoter employed are inflexible. Abscisic acid (ABA) is a plant hormone involved in stress adaptation and seed development and acts in part by regulating gene expression through a combinatorial network of ABA-specific transcription factors. The ABA INSENSITIVE-5 (ABI5) basic leucine zipper (bZIP) transcription factor and closely related ABI-5-like homologues have been tested for transactivation of the ABA-inducible wheat Em promoter in transiently transformed rice and maize protoplasts. The functional interactions of co-expressed ABI5 and the ABI5-like ABA-Response Element-Binding Factors (ABF1, ABF2, ABF3, ABF4, AREB3, and DPBF4), which have highly conserved domains (C1-C3, bZIP), were tested with each other and with co-expressed maize VIVIPAROUS-1 (VP1) transcription factor. Overexpressed Arabidopsis ABI5, ABF3, ABF4, AREB3, DPBF4, and maize VP1, but not ABF1 and ABF2, individually show synergy with ABA in rice embryonic and/or maize mesophyll protoplasts. However, when ABI5 and ABF3 are co-expressed in rice protoplasts, they show no functional interaction with each other, in contrast to strong synergy observed with ABI5, or ABF3 co-expressed with VP1 in rice or maize. Furthermore, AREB3, DPBF4, and ABF4 do not work in synergy with co-transformed maize VP1, whereas ABI5, ABF3 and ABF1 can synergize with VP1 in maize mesophyll protoplasts. This latter result is in contrast to the observation that ABF1 does not appear to interact with ABA alone. Taken together, these functional data provide several examples to support our claim that formulations of ABI5-like and VP1-like transcription factors will provide novel and useful means to effect stress- and ABA-inducible gene expression that in turn will enhance valuable traits (productivity, yields, stress tolerance) of commercial varieties of plants. These data provide a proof-in-principle that expression of transgenes from various plant species in target tissues such as leaves or seeds may render transgenic horticultural and ornamental plants and crops to be better able to withstand environmental stress via coordinated regulation of multiple endogenous gene sets in stress tolerance pathways. We have demonstrated that over-expression of maize VP1, Arabidopsis ABI5, and several but not all ABI5-related family members transactivates various ABA-inducible promoters from both monocots and dicots in rice or maize protoplasts, proving that these transcription factors are key targets of a conserved ABA signaling pathway in plants. Others have shown that ectopic expression of ABI3, ABI4, or ABI5 transcription factors results in ABA hypersensitivity of vegetative tissues which is partly dependent on increased ABI5 expression. Taken together, these results show that these transcription factors participate in combinatorial control of gene expression, by forming a regulatory complex mediating seed-specific and/or ABA-inducible expression.

### BRIEF DESCIPTION OF THE DRAWINGS

The features and advantages of the present invention will become apparent from the following detailed description of a preferred embodiment thereof, taken in conjunction with the accompanying drawings, in which:

FIG. 1. Overexpression of ABF3 and ABF4 is sufficient to transactivate ABA-inducible Em-GUS expression in rice protoplasts. Protoplasts were transformed with Em-GUS and a “dummy” effector construct (pD2.6) or co-transformed with a Ubi-ABF construct and treated with or without 100 μM ABA. The values are the average ±S.E.M. of four replicate transformations.

FIG. 2. Overexpressed ABF3 interacts synergistically with ABA and VP1, but not ABI5. Rice protoplasts were transformed with either Em-GUS alone or in pairwise combinations of Ubi-ABF3, Ubi-ABI5, or 35S-VP1. The values are the average ±S.E.M. of four replicate transformations.

FIG. 3. AREB3, or DPBF4 overexpression in maize mesophyll protoplasts is sufficient to transactivate Em-GUS expression, while ABF1, but not AREB3 or ABF4, can synergize with co-transformed VP1. The values are the average ±S.E.M. of four replicate transformations.

### DETAILED DESCRIPTION OF THE INVENTION

Strong and novel activities in rice embryonic and maize mesophyll protoplasts of the transcription factors VP1, ABI5, ABF1, ABF3, ABF4, AREB3 and DPBF4 genes from maize and Arabidopsis have been demonstrated. Taken together, the data suggest that transgenic plants that ectopically express VP1, ABI3 or orthologues from various species in combination with ABI5-like family members will respond to abiotic stressors (e.g. salt, drought, cold) by activating ABA-inducible target (transgene) promoters as well as endogenous promoters that coordinate expression of genes involved in stress adapation (e.g. LEA and COR genes). The multi-component expression system demonstrated herein could be expanded in the future by modifying the regulatory sequences of the target promoter or the promoter driving the VP1 or ABI5-like effector to include tissue-specific enhancer elements or stress-response elements to direct the expression of any target gene of interest. It is known in animal systems that targeting of some combinations of transcription factors to the same promoter may produce synergistic effects on the expression level. The strategy disclosed herein has the potential to amplify the expression level from a promoter with desirable tissue- or cell specificity. For example, recent results have shown that grain-filling in rice is critically dependent on water status and ABA levels, suggesting that amplification of ABA response pathways by ectopic transcription factor expression in appropriate tissues and at critical times during development could have beneficial effects. The multi-component benefits of transcription factor synergy could be realized by genetic crossing of two lines harboring separate transcription factor components. These transgenic materials would also be useful resources for further work on the cell biology and functional genomics of ABA- and tissue-specific gene regulation and signal transduction.

ABA signaling pathways are highly conserved among monocots and dicots, suggesting that a multicomponent transgenic approach to engineering stress tolerance with effectors from diverse plant species is practical. Evidence presented here with overexpressed Arabidopsis ABFs, AREB3, DPBF4, ABI5 and maize VP1 in rice embryonic and maize mesophyll protoplasts further extends this claim. Based on our results and the results of others in the field, who showed that maize VP1 was functionally redundant with ABI3, other orthologues of VP1/ABI3 could substitute for VP1 in a multicomponent heterologous transactivation system. Consistent with this, expression of a dicot GAI orthologue in transgenic rice resulted in desirable dwarfing traits, suggesting that heterologous regulatory genes can be used to affect traits in a wide range of crop species. Transgenic rice plants that express the maize phosphoenolpyruvate carboxylase (PEPC) and pyruvate orthophosphate dikinase (PPDK) exhibit a higher photosynthetic capacity (up to 35%) than untransformed plants, mainly associated with an enhanced stomatal conductance and a higher internal CO₂ concentration. An additional benefit of using heterologous genes in such a multicomponent system is that they may minimize artifacts such as cosuppression.

One potential drawback to overexpressing regulatory factors that confer stress tolerance to transgenic crops is reduced yields through pleiotropic “knock on” effects that indeed may be the direct consequence of stress adaptation mechanisms triggered by the transgene effector. In this scenario, the present method would still find application in horticultural crops where yields, per se, may be less important. Likewise, in ornamental species the slow-growth, stress-adapted phenotype would be a value-added trait.

Some novel activities of ABI5-like family members and ABI5 derivatives alone and in combination with maize VP1 have been demonstrated. Ectopic or controlled (e.g. inducible) expression of these and related effectors in any plant species will result in conditionally altered stress responses and possibly higher levels of engineered target gene expression than otherwise possible. Given the efficacy of ABA in regulating such basic processes as seed development, dormancy vs. germination, transpiration and stress responses, the present invention and research can pave the way to important biotechnological applications.

### EXAMPLE

Materials and Methods

Plant Materials. Maize mesophyll protoplasts were isolated from 20-hr illuminated leaves of 10 day old maize seedlings that had been kept in the dark at 25° C. The middle part of the second leaves (about 6 cm in length) was cut into 0.5 mm strips with a razor blade and digested in an enzyme solution containing 1% (w/v) cellulose RS, 0.1% (w/v) macerozyme R10 (Yakult Honsha, Nishinomiya, Japan), 0.6 M mannitol, 10 mM MES (pH 5.7), 1 mM CaCl₂, 1 mM MgCl₂, 10 mM β-mercaptoethanol, and 0.1% BSA (w/v) for 3 hr at room temperature. Protoplasts were released by shaking on a rotary shaker at 80 rpm for 10 min and were filtered through a 70 μm nylon filter. Protoplasts were collected by centrifugation at 100 g for 2 min, washed in cold 0.6 M mannitol solution, centrifuged, and resuspended at 2×10⁶/mL in cold 0.6 M mannitol. Electroporation conditions were 400 V/cm, 200 μF, 10 msec, and two pulses with a Biorad GenePulser apparatus. Each sample contained 3×10⁶ protoplasts and about 50 μg DNA in 0.3 mL of 0.6 M mannitol and 20 mM KCl.

Embryonic rice (Oryza sativa) callus cultures (Radon 6 from the International Rice Research Institute, Los Baños, Phillipines) were obtained from Dr. Tom Hodges, Purdue University, West Lafayette, Ind., 47909, USA. Embryonic rice callus cultures were grown as suspensions in liquid culture as well as on phytagel plates containing MS medium supplemented with 2.0 mg/L 2,4-D. Cultures were propagated and digested for making protoplasts as previously described except that 10 mM HEPES (Sigma, St. Louis, Mo., USA), pH 5.6, was substituted for phosphate in the Krens' F medium, and 2% (weight/volume; w/v) cellulase YC, 0.35% (w/v) macerozyme, and 0.1% (w/v) pectolyase Y23 were used for overnight digestion (Karlan Research Products, Santa Rosa, Calif., USA). Protoplasts were transformed with various mixtures of DNA reporter and effector constructs using polyethylene glycol precipitation. Transformed protoplasts were incubated with or without 100 μM ABA for 16 h in the dark in Krens solution before quantifying β-glucuronidase (GUS) and luciferase (LUC) reporter enzyme activities as previously described. ABA was dissolved and stored in absolute ethanol at −20° C. as a 0.1 M stock solution. Prior to use, required dilutions of ABA were made in Krens solution, and control samples received the same volume of solvent as in ABA treatments.

Plasmid Constructs

Plasmid pBM207 contains the wheat (Triticum aestivum) Early Methionine-labeled (Em) promoter driving the expression of GUS, encoded by uidA from Escherichia coli. pDH359 contains ABI5 cDNA driven by Ubiquitin promoter. Plasmid pCR349.13S contains the 35S promoter driving the VP1 sense cDNA. Plasmid pDirect2.6 contains the Ubi promoter in a reverse orientation and was used as control construct to balance the total amount of input plasmid DNA between various treatments. Plasmid pAHC18 contains the Ubi promoter driving firefly (Photinus pyralis) LUC cDNA and was included in transformations to provide an internal reference for non-ABA-inducible transient transcription in reporter enzyme assays. ABF1-ABF4, AREB3, and DPBF4 were amplified by PCR using gene-specific primers from an Arabidopsis cDNA library and were cloned into plasmid pDH349 containing the maize Ubiquitin promoter. Primers used for PCR amplification are listed in Table I.

TABLE I

Gene-specific PCR primers used to clone
Arabidopsis ABI5-like cDNAs used herein.

| | |
|---|---|
| | Primer sequence (5′->3′; |
| Gene | F = forward, R = reverse) |

| | |
|---|---|
| ABF1 | |
| SEQ ID NO 1 | F: cccaagcttggatccaaagggtctgattcgtttgt |
| SEQ ID NO 2 | R: cggggtaccgttaacgtcacatcttctctatagct |
| ABF2 | |
| SEQ ID NO 3 | F: cccaagcttggatcccccaaacgaagaaccaaaca |
| SEQ ID NO 4 | R: cggggtaccgatatcttcttcaaaattggtaactc |
| ABF3 | |
| SEQ ID NO 5 | F: ccgctcgagggatccgaagcttgatcctcctagtt |
| SEQ ID NO 6 | R: cggggtaccgatatcagatacaagataaattcact |
| ABF4 | |
| SEQ ID NO 7 | F: cccaagcttggatccgaacaagggttttagggctt |
| SEQ ID NO 8 | R: cggggtaccgatatcgttgccactcttaagtaata |
| AREB3 | |
| SEQ ID NO 9 | F: cccactagtggatccatggattctcagaggggtat |
| SEQ ID NO 10 | R: cggggtaccgatatctcagaaaggagccgagcttg |
| DPBF4 | |
| SEQ ID NO 11 | F: cccggtaccggatccacagtttctaaggcaaaata |
| SEQ ID NO 12 | R: cggaggcctgaattcacttgaactagtgtttgtac |

Results

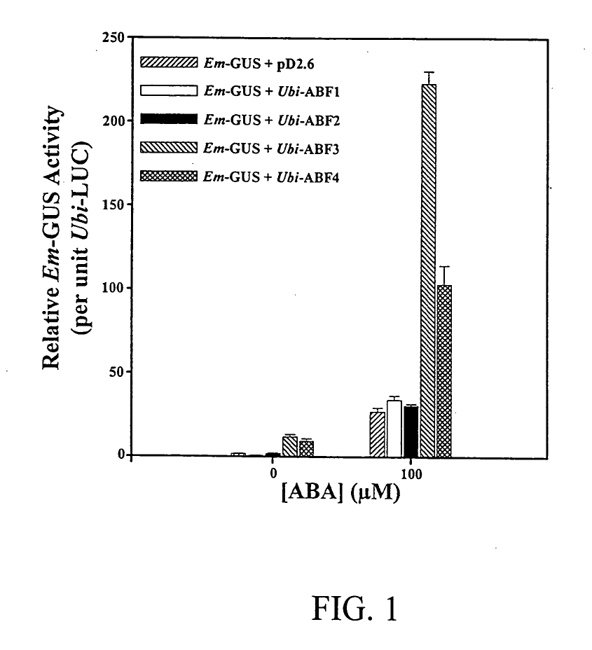

Previous results demonstrated that overexpressed ABF1 and ABF3 had positive effects on ABA-inducible Em-GUS reporter gene expression in transiently transformed rice protoplasts, providing functional evidence for the involvement of these proteins in ABA- and stress signal transduction. Simultaneously, it has been shown that ABF3 and ABF4 overexpression in transgenic Arabidopsis results in ABA hypersensitivity and other ABA-associated phenotypes such as altered ABA-inducible gene expression, reduced transpiration, and enhanced drought tolerance. The functional roles of the ABI5-like family members ABF1-ABF4 in regulation of ABA-inducible gene expression in rice protoplasts were tested and the results are shown in FIG. 1. Consistent with previous results, overexpressed ABF1 and ABF3 had slight and strong synergy with exogenous ABA, respectively. However, overexpression of ABF2 had no effect on Em-GUS expression, see FIG. 1. Interestingly, overexpression of ABF3 or ABF4 was sufficient for transactivation of the Em promoter. These results are consistent with those of previous researchers who showed that overexpressed ABF3 or ABF4 resulted in accumulation of the LEA genes rd29A and rab18.

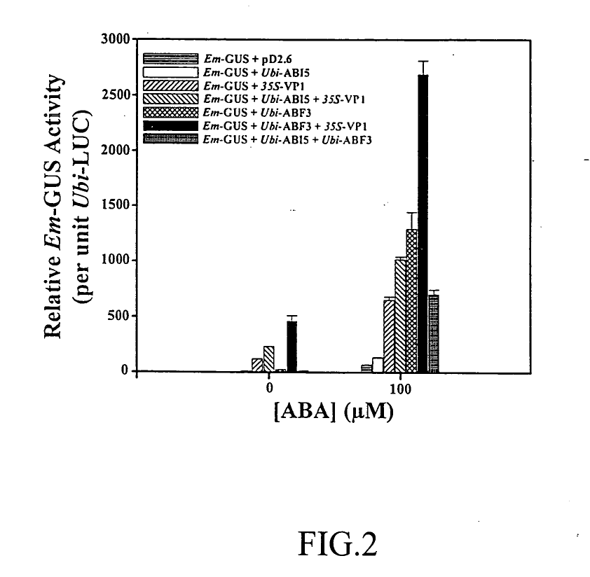

FIG. 2 displays the results of an ABA-inducible reporter gene expression experiment with transiently-transformed rice protoplasts overexpressing ABI5, ABF3 (a related member of the ABI5-family of bZIP transcription factors), and VP1 transcription factors alone and in pairwise co-transformations. As previously reported ABI5, ABF3, and VP1 transactivated the Em promoter and acted in synergy with ABA. More importantly, ABF3 and VP1 synergized with each other and with ABA when co-expressed. This activity was about twice the synergistic activity seen between ABI5 and VP1. However, paired expression of ABF3 and ABI5 in protoplasts did not result in synergy. Based on these two examples of ABI5-family member synergy with maize VP1, it is suggested that all members of the ABI5 bZIP family will have functional interactions with VP1 and ABI3, including orthologues from various species.

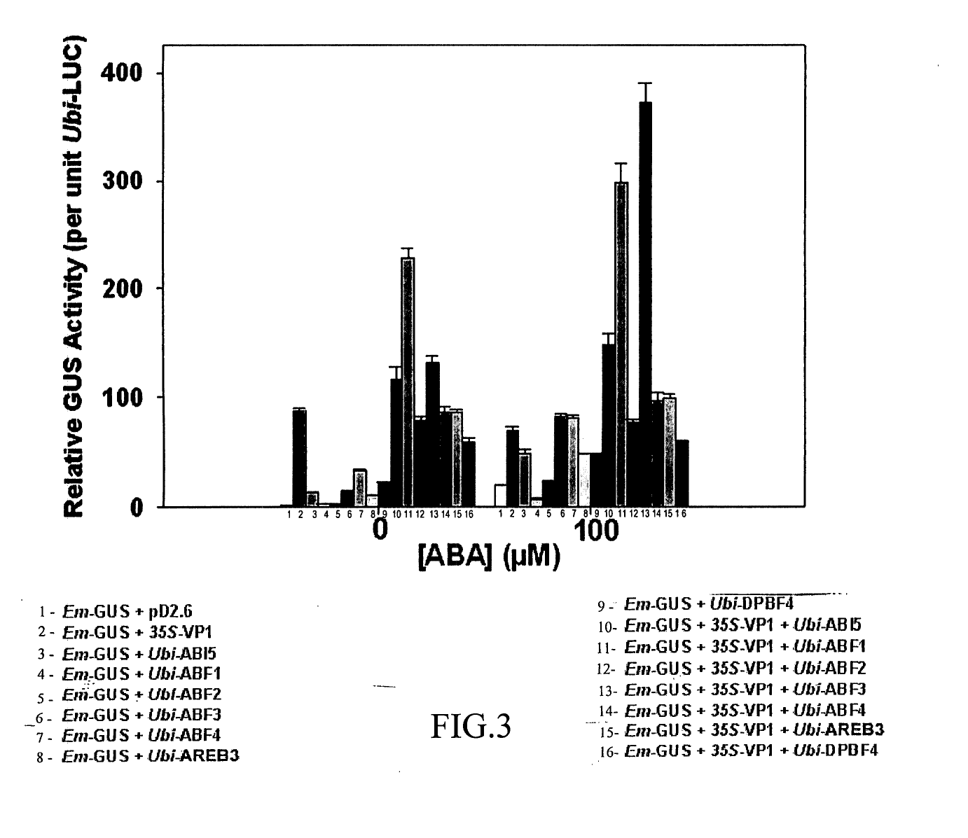

To provide evidence in support of the hypothesis that transcriptional regulation of ABA signaling is highly conserved among higher plants and in different tissue types, various ABI5-like homologues from Arabidopsis were tested for their ABA signaling activites and functional interactions with VP1 in maize mesophyll protoplasts. The results are shown in FIG. 3. As previously shown for rice embryonic protoplasts (see FIGS. 1 and 2), ABI5, ABF3 and ABF4 synergize with exogenous ABA and are sufficient for transactivation of the Em promoter when overexpressed in maize mesophyll protoplasts. ABF 1 and 2 have lower levels of ABA synergy compared to ABF3 and ABF4 in maize, similar to activities observed in rice (see FIG. 1). Furthermore, the ABI5-like family members AREB3 and DPBF4 synergize with exogenous ABA and are sufficient when overexpressed to transactivate the Em promoter (see FIG. 3). This finding demonstrates that ABI5-like family members can function in ABA signaling and suggests they may have novel/unique functions and activities including interactions with VP1 and VP1-like homologues. Indeed, ABF1 showed a strong synergy with VP1 in maize despite showing little synergy with ABA, unlike ABI5 and ABF3, which showed similar interactions with ABA and VP1 as seen in rice (compare FIGS. 2 and 3). Interestingly, ABF4, AREB3, and DPBF4 showed no functional interactions with VP1 (see FIG. 3) in maize mesophyll protoplasts. Therefore, both similarities and differences are seen in the functional interactions of Arabidopsis ABI5-like genes with VP1. We speculate that other VP1-like family members of Arabidopsis (of which there are over 20) may be the cognate partners of ABI5-like family members such as ABF2 and ABF4 that may regulate distinct, or tissue-specific aspects of ABA and stress signaling.

Although the present invention has been disclosed in terms of a preferred embodiment, it will be understood that numerous additional modifications and variations could be made thereto without departing from the scope of the invention as defined by the following claims:

1. A method of enhancing stress-related traits in plants comprising,
a) regulating absiscic acid- and stress-inducible gene expression using ABI-5-like and Viviparous-1-like transcription factors.


SEQUENCE LISTING (12 sequences)

SEQ ID NO: 1 - DNA, 35 bp, Arabidopsis thaliana
cccaagcttggatccaaagggtctgattcgtttgt

SEQ ID NO: 2 - DNA, 35 bp, Arabidopsis thaliana
cggggtaccgttaacgtcacatcttctctatagct

SEQ ID NO: 3 - DNA, 35 bp, Arabidopsis thaliana
cccaagcttggatcccccaaacgaagaaccaaaca

SEQ ID NO: 4 - DNA, 35 bp, Arabidopsis thaliana
cggggtaccgatatcttcttcaaaattggtaactc

SEQ ID NO: 5 - DNA, 35 bp, Arabidopsis thaliana
ccgctcgagggatccgaagcttgatcctcctagtt

SEQ ID NO: 6 - DNA, 35 bp, Arabidopsis thaliana
cggggtaccgatatcagatacaagataaattcact

SEQ ID NO: 7 - DNA, 35 bp, Arabidopsis thaliana
cccaagcttggatccgaacaagggttttagggctt

SEQ ID NO: 8 - DNA, 35 bp, Arabidopsis thaliana
cggggtaccgatatcgttgccactcttaagtaata

SEQ ID NO: 9 - DNA, 35 bp, Arabidopsis thaliana
cccactagtggatccatggattctcagaggggtat

SEQ ID NO: 10 - DNA, 35 bp, Arabidopsis thaliana
cggggtaccgatatctcagaaaggagccgagcttg

SEQ ID NO: 11 - DNA, 35 bp, Arabidopsis thaliana
cccggtaccggatccacagtttctaaggcaaaata

SEQ ID NO: 12 - DNA, 35 bp, Arabidopsis thaliana
cggaggcctgaattcacttgaactagtgtttgtac